(19) United States
(12) Patent Application Publication
(10) Pub. No.: US 2010/0048921 A1
(43) Pub. Date: Feb. 25, 2010

(54) PHARMACEUTICALLY ACTIVE COMPOUNDS

(76) Inventors: Herbert Görne, Hamburg (DE); Lutz Müller-Kuhrt, Potsdam (DE); Christian Mang, Berlin (DE)

Correspondence Address: BUCHANAN INGERSOLL & ROONEY PC, P.O. BOX 1404, ALEXANDRIA, VA, 22313-1404, US

(21) Appl. No.: 12/522,770

(22) PCT Filed: Jan. 10, 2008
(86) PCT No.: PCT/DE2008/000040

§ 371 Date: Jul. 14, 2009

(30) Foreign Application Priority Data
Jan. 10, 2007 (DE) 10 2007 002 386.5

(51) Int. Cl. C07D 311/02

(52) U.S. Cl. 549/403

(57) ABSTRACT

This invention relates to pharmaceutically active compounds consisting of an isoflavone or isoflavone-glycoside and an amino acid, a peptide or peptide derivate with 2 to 5 amino acids which is covalently bonded to these, as well as pharmaceutically acceptable salts or solvates of these compositions as well as to the use of those substances for the production of pharmaceutical compositions, in particular for the treatment of thrombocyte aggregation and tumor therapy.

Consequently, the areas of application of the invention are medicine and the pharmaceutical industry.

The compounds according to the invention have the general formula (I).

X-Pep (I)

whereupon
X is an isoflavone or isoflavone-glycoside, and
Pep is an amino acid or a peptide or peptide derivate with 2 to 5 amino acids,
and there is a bond between X and Pep either with a covalent bond or with a linker system.

This invention relates to pharmaceutically active compounds consisting of an isoflavone or isoflavone-glycoside and an amino acid, a peptide or peptide derivate with 2 to 5 amino acids which is covalently bonded to these, as well as pharmaceutically acceptable salts or solvates of these compositions as well as to the use of those substances for the production of pharmaceutical compositions, in particular for the treatment of thrombocyte aggregation and tumor therapy.

Consequently, the areas of application of the invention are medicine and the pharmaceutical industry.

Isoflavones which are occasionally also called isoflavonoids form a group of mostly yellow-coloured plant pigments which are derived from isoflavone. Within the frame of this invention, the isoflavones also include their respective glycosides. The most well-known isoflavones in particular include isoflavone, daidzein, genistein, prunetin, biochanin A, orobol, santal, glycitein, pratensein, formononetin, genistin, 6″-O-malonylgenistin, 6″-O-acetylgenistin, daidzin, 6″-O-malonyldaidzin, 6″-O-acetyldaidzin, glycitin, ononin and sissotrin and also genistin, daidzin, 6″-O-malonylglycitin and 6″-O-acetylglycitin. The malonyl-glycosides of the genistein make up the majority of the isoflavones in soybeans. In fermented soy products, the isoflavones are mainly present in their respective aglycon form genistein, daidzein and glycitein.

Some isoflavones are known for having an estrogen effect, for example on grazing animals. Some other isoflavones are supposed to have an antioxidant effect within humans or other mammals but such effect could not be proven until now. It is also controversial whether isoflavones have an anti-carcinogenic, anti-atherogenic, anti-osteoporotic and/or hypolipidemic effect and if yes, which ones do. In many cases, it has not been possible until now to reproduce the effects attributed to the isoflavones by administering pure isoflavone, if necessary with the common pharmaceutical carrier and auxiliary substances. In particular, it was not possible to achieve an antioxidant effect of genistein despite the administration of high doses and the proof of a high concentration of genistein in the target cells of a human being.

For example, it is known that isoflavones, and in particular genistein, inhibit thrombocyte aggregation depending on the dosis (1). In doing so, the influence of the isoflavone on the function of the thrombocytes is of a complex nature: On the one hand, in particular genistein blocks various membrane receptors (adenosine receptor, von Willebrand factor) (2, 3), and because of its characteristic chemical structure in particular also the thromboxane A2 receptor (4). On the other hand, genistein inhibits thrombocyte aggregation with the modulation of intracellular metabolic processes: inhibition of tyrosine kinases in the metabolism of cyclooxygenase (5), modulation of cAMP with the inhibition of phosphodiesterase (6), reduction of the intracellular availability of peroxide as an important agent of phospholipase C and the arachidonic acid metabolism (7, 8). Genistein is an isoflavone obtained from soy. Different surveys show an inhibiting effect of genistein on the growth of malignant cells such as for example those of haematopoietic tumors such as leukaemia and lymphoma, melanoma and epithelial tumors such as breast, lung, prostate and head and neck squamous cell carcinoma (1-5).

The growth inhibiting activity of geinstein is based on its antiproliferative and proapoptotic effect (6). Because of the restricted cellular uptake of genistein in cell culture systems, this effect however only appeared with relatively high concentrations which can hardly be achieved in vivo (7).

It has already been proposed to produce synergistic pharmaceutical compositions of isoflavones or isoflavone-glycosides with short-chain peptides (PCT/DE2006/001795). In these compositions, the availability of isoflavones was considerably increased because of the better cellular uptake. However, for this, comparatively high concentrations of substrate—in particular peptide—are necessary. On the one hand, in case of oral application, the peptides used are partially broken down with the colon transit, on the other hand, the plasma half-times for peptides amount to minutes which reduces the availability and causes undesired side-effects. For example, such side-effects have been described for glutamic acid (as a degradation product of glu-glu) in the sense of central nervous disorders. As a consequence, the precise controllability of plasma levels after the application which is absolutely necessary for the therapy of certain diseases cannot be reached.

Therefore, it was the task of this invention to relieve or at least alleviate the disadvantages described above. In particular, the task was to provide compounds which can especially be used as a pharmaceutical in the sense of a genistein derivate in addition to various possibilities of use, whereupon an improved intracellular availability should for example lead to an inhibition of thrombocyte aggregation which is better than with genistein and/or the compositions of isoflavones and short-chain peptides or an improved anti-tumor activity. As far as possible, additional areas of application of isoflavones should also be opened.

This task is solved with compounds in accordance with claim 1, pharmaceutical compositions in accordance with claim 10 and the use of such compounds in accordance with one of the claims 11 or 14-16. The other claims are preferred versions and embodiments of the invention.

Absolutely surprisingly, it has been found that compounds according to formula (I),

X-Pep (I),

in which X is an isoflavone or isoflavone-glycoside and Pep is an amino acid or a peptide or peptide derivate with 2 to 5 amino acids, and there is a covalent bond between X and Pep or the bond is ensured with a suited linker system (such as ethylene glycol, ethanolamine, higher homologs thereof or the respective polyethylene glycol derivates) as well as pharmaceutically acceptable salts or solvates of these compositions increase the effects of the isoflavone component of the compound on organisms, in particular mammals or only cause the development of the biological effects.

In preferred forms, the component X of the compound I consists of an isoflavone or isoflavone-glycoside, a substance of the general formula (II) or a pharmaceutically acceptable salt or solvate of such a substance:

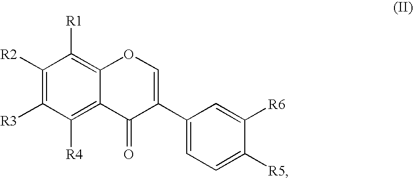

whereupon the residuals R1, R2, R3, R4, R5 and R6 can autonomously have the following meanings: hydrogen, hydroxy, methoxy or glycoside (Glc), whereupon one of the residuals 1-6 in compound I is replaced by the residual Pep (meaning explained above). Preferred places for links are R2 and R5.

The glycoside in formula II has the general formula III

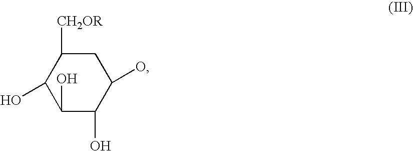

whereupon R is selected from the group consisting of hydrogen, acetyl and malonyl independent of R1, R2, R3, R4, R5 and R6.

Especially preferred are those compounds in accordance with the invention in which the component X consists of isoflavone, daidzein, genistein, prunetin, biochanin A, orobol, santal, glycitein, pratensein, formononetin, genistin, 6″-O-malonylgenistin, 6″-O-acetylgenistin, daidzin, 6″-O-malonyldaidzin, 6″-O-acetyldaidzin, glycitin, ononin or sissotrin.

| Trivial name | R1 | R2 | R3 | R4 | R5 | R6 | R |
|---|---|---|---|---|---|---|---|
| Isoflavone | H | H | H | H | H | H | — |
| Daidzein | H | OH | H | H | OH | H | — |
| Genistein | H | OH | H | OH | OH | H | — |
| Prunetin | H | OCH₃ | H | OH | OH | H | — |
| Biochanin A | H | OH | H | OH | OCH₃ | H | — |
| Orobol | H | OH | H | OH | OH | OH | — |
| Santal | H | OCH₃ | H | OH | OH | OH | — |
| Glycitein | H | OH | OCH₃ | H | OH | H | — |
| Pratensein | H | OH | H | OH | OCH₃ | OH | — |
| Formononetin | H | OH | H | H | OCH₃ | H | — |
| Genistin | H | Glc | H | OH | OH | H | H |
| 6″-O- | H | Glc | H | OH | OH | H | COCH₂COOH |
| malonylgenistin | | | | | | | |
| 6″-O- | H | Glc | H | OH | OH | H | COCH₃ |
| acetylgenistin | | | | | | | |
| Daidzin | H | Glc | H | H | OH | H | H |
| 6″-O- | H | Glc | H | H | OH | H | COCH₂COOH |
| malonyldaidzin | | | | | | | |
| 6″-O- | H | Glc | H | H | OH | H | COCH₃ |
| acetyldaidzin | | | | | | | |
| Glycitin | H | Glc | OCH₃ | H | OH | H | H |
| Ononin | H | Glc | H | H | OCH₃ | H | H |
| Sissotrin | H | Glc | H | OH | OCH₃ | H | H |

In other preferred versions, the component X contains two to three OH groups which are free of or completely or partially substituted by monosaccharides, including monosaccharides which are partially acylated with acetic acid, malonic acid, cinnamic acid, coumaric acid, caffeic acid or ferulic acid, or by disaccharides, including disaccharides which are partially acylated with acetic acid, malonic acid, cinnamic acid, coumaric acid, caffeic acid or ferulic acid, or by methyl or sulphate.

In an especially preferred version, X consists of genistein, genistin, daidzein and/or daidzin, whereupon genistein is preferred.

The peptide residual of the compound X-Pep (I) preferably consists of a peptide or peptide derivate with 2 to 5 amino acids, whereupon at least 2 amino acids include side-chains which are negatively loaded with pH=7.

In preferred versions, the component Pep consists of a dipeptide which is selected from the group of peptides with the sequences DD, EE, DE and ED, whereupon D=aspartic acid and E=glutamic acid. Such short peptide components and their pharmaceutically acceptable salts or solvates have surprisingly proven to have a strong synergetic effect, in particular in combination with genistein, genistin, daidzein and daidzin. Of the named dipeptides, glutamic acid dipeptide (a peptide of the sequence EE) is especially preferred. With glutamic acid dipeptide in connection with geinstein, an extremely remarkable increase of the thrombocyte aggregation inhibiting effect and of the inhibition of tumor growth in comparison to genistein could be observed with humans in vitro and in vivo.

In other preferred versions, the component Pep consists of a peptide which is selected from the group of peptides with the sequences

### DDDDD, DDDDE, DDDED, DDDEE, DDEDD, DDEDE, DDEED, DDEEE, DEDDD, DEDDE, DEDED, DEDEE, DEEDD, DEEDE, DEEED, DEEEE, EDDDD, EDDDE, EDDED, EDDEE, EDEDD, EDEDE, EDEED, EDEEE, EEDDD, EEDDE, EEDED, EEDEE, EEEDD, EEEDE, EEEED, EEEEE,

### DDDD, DDDE, DDED, DDEE, DEDD, DEDE, DEED, DEEE, EDDD, EDDE, EDED, EDEE, EEDD, EEDE, EEED, EEEE,

### DDD, DDE, DED, DEE, EDD, EDE, EED, EEE;

whereupon D=aspartic acid and E=glutamic acid.

In principle, any possible combinations of X and Pep which are covalently bonded are suited as a compound in accordance with the invention. For example, for different areas of use, a compound in which the isoflavone genistein is covalently bonded with the peptide or the peptide derivate of the sequence EE, whereupon E=glutamic acid has proven to be effective.

Principally, the covalent bond between X and Pep can be arbitrarily formed within a compound according to the invention, but a carbamate, ether or ester bond is preferred. A version in which the peptide is directly linked to a hydroxyl group of X which is located at one of the positions R1-R6 via its N-terminal amino group or its C-terminal carboxyl group or the primary hydroxyl function which can be gained thereof by reduction with the formation of a covalent carbamat, ether or ester bond or connects the two functionalities via a suitable linker system has proven to be particularly effective. Ethylene glycol, ethanolamine, higher homologs thereof or the respective polyethylene glycol derivates are suited as linker systems.

In a particularly favourable version, the covalent bond consists of an amide, an ether or a carboxylic acid ester, preferably if the bond takes place via R2 or R5 of X.

The present invention furthermore includes the use of the named compounds or of pharmaceutically acceptable salts or solvates thereof for the production of pharmaceutical compositions for the treatment of and/or the protection against medical indications of the human organism or other mammals' organisms.

With this, preferred medical indications include the prophylaxis and therapy of thromboembolic diseases, cardiovascular diseases, vascular diseases and vascular anomalies, diseases connected with an increased number of thrombocytes/thrombocyte aggregation, metabolic disorders and cancer diseases.

Especially preferred medical indications include the prophylaxis and therapy of: hypertonia, hypercholesterolaemia, heart attack and reinfarction, infarcts and reinfarctions of other organs, apoplexy, pulmonary embolism, leg vein thrombosis, arteriosclerotic angiopathy, diseases caused by an increased number of thrombocytes and/or aggregation, diabetes mellitus, hyperhomocysteinaemia, malignant tumors and/or osteoporosis as well as the pre and postoperative prophylaxis of thrombosis.

The present invention furthermore relates to the use of the named compounds and active substances combinations for the production of pharmaceutical compositions for the treatment of and/or the protection against medical indications of the human organism or other mammals' organisms.

In addition to this, the present invention includes the use of at least one compound in accordance with the invention or of pharmaceutically acceptable salts or solvates thereof as an additive to pharmaceutical compositions in order to improve the availability of active substances in these pharmaceutical compositions in mammals.

The present invention particularly includes the use of the named compounds or pharmaceutically acceptable salts or solvates thereof as a nutritional additive, as an additive for the protection of cells in fermenters or bioreactors, as a nourishment for animals and as a pesticide.

In particular, the use of the compounds in accordance with the invention or pharmaceutically acceptable salts or solvates thereof is preferred for the reduction of human thrombocyte aggregation or of the growth of malignant cells of humans such as those of haematopoietic tumors such as leukaemia and lymphoma, solid tumors such as melanoma, epithelial tumors such as breast, lung, gastrointestinal (e.g. pancreas), and in particular gastric, colon and prostate and head and neck squamous carcinoma.

Furthermore, the present invention includes procedures for the production of the compound I

including the following steps:
a) provision of an active substance selected from the group which consists of isoflavone, daidzein, genistein, prunetin, biochanin A, orobol, santal, glycitein, pratensein, formononetin, genistin, 6″-O-malonylgenistin, 6″-O-acetylgenistin, daidzin, 6″-O-malonyldaidzin, 6″-O-acetyldaidzin, glycitin, ononin and sissotrin and/or a pharmaceutically acceptable solvate or salt of the active substance, whereupon the active substance shows a protected side group, if necessary,
b) provision of a peptide or peptide derivate with a length of 2 to 5 amino acids or provision of an amino acid and/or a solvate or salt of the peptide or of the amino acid, whereupon at least one of the amino acids disposes of a side group which is protected and the N-terminus of the peptide or the amino acid is protected and/or the C-terminus of the peptide or of the amino acid is connected to a solid phase,
c) mixing of the active substance of a) with the active substance of b) and an activation reagent, preferably for the activation of carboxyl or hydroxyl groups.

The production is for example achieved with

a)
1. production of a peptide of a suitable length and sequence with at least one (OtBu)-protected side group and Boc-protected N-terminus via solid phase synthesis at the resin and elimination from the resin.
2. coupling of X to the protected dipeptide with HATU.
3. tBu/Boc-deprotection.
4. cleaning.
   b)
1. production of a peptide of a suitable length and sequence with at least one (OtBu)-protected side group and Boc-protected N-terminus via solid phase synthesis at the resin and elimination from the resin.
2. coupling of BocX to the protected dipeptide with HATU.
3. tBu/Boc-deprotection.
4. cleaning.
   c)
1. production of a peptide of a suitable length and sequence with at least one (OtBu)-protected side group and Fmoc-protected N-terminus via solid phase synthesis at the resin and subsequent Fmoc fission.
2. realisation with BocX and triphosgene.
3. elimination of resin.
4. tBu/Boc-deprotection.
5. cleaning.
   or d)
0. coupling of an N-terminal Fmoc-protected amino acid to a resin, whereupon the amino acid shows an allyl ester-protected side group,
1. A) fission of the allyl ester at the resin. B) coupling of the released acid group with BocX upon utilisation of HATU.
2. A) Fmoc fission. B) if necessary coupling of at least one other, preferably N-terminal Boc-protected amino acid which shows a tBU-protected side group to the solid phase-bound N-terminus.
3. elimination of resin.
4. tBu/Boc-deprotection.
5. cleaning.
   or e)
1. production of a peptide of a suitable length and sequence with at least one (OtBu)-protected side group and Fmoc-protected N-terminus via solid phase synthesis at the resin and subsequent Fmoc fission.
2. production of an ether-linked ethanolamine-X-conjugate.
3. coupling of the two fragments.
4. deprotection.
5. cleaning.

The starting point of the invention is the surprising finding that the new compounds of the formula (I) are transported to the cell via a cellular peptide transport system in a considerably more effective way. This guarantees that even low concentrations of these compositions can trigger physiological effects, in particular with the prevention, treatment and relief of human thrombocyte aggregation and tumors and the indications related to these. Thus, considerably lower amounts are necessary than with the use of the corresponding pure isoflavone or isoflavone-glycoside.

In particular, the compound components X and Pep are synergistically effective due to their covalent bond in a favourable way. In this, additional synergy effects with additional active substances may occur.

In accordance with the invention, active substance relates to a substance which might cause a pharmaceutically desired change of the physiological state of the treated mammal, and especially a human being, when it is administered. Active substance especially relates to the pharmaceutically effective component of a medicine, in particular the active substance component X of the compound according to the invention. If substances are named in their singular form within the frame of this invention, this also relates to mixtures of several of the respective substances if nothing else is specified. Therefore, the invention also relates to pharmaceutical compositions the active substance of which is a mixture of two or several compounds of the formula (I). Moreover, according to the invention, an active substance and a peptide and/or compounds according to the invention which were developed from them by covalent bonds also include their respective pharmaceutically acceptable salts or solvates.

Within the frame of the present invention, the term peptide relates to linear or branched peptides which can consist of the 20 gencoded amino acids as well as of the non-naturally appearing alpha, beta and gamma amino acids. Within the frame of the present invention, peptide derivate relates to a peptide which is modified by at least one linear, cyclic or branched alkyl, alkyl ether, alkylthioether, alkoxy, acyl or aryl residue which is substituted by halogen, hydroxy or amine or non-substituted, saturated or aliphatic in the main and/or the side-chain, whereupon the residue contains between 1 and 20 carbon atoms.

The pharmaceutical and/or therapeutic effect reached with the composition especially corresponds to the effect of an antioxidant if the active substance and/or the active substance component X consist of an isoflavone or an isoflavone-glycoside. The compound in accordance with the invention for the first time makes it possible to achieve the antioxidant effect of an isoflavone which has only been supposed or described with high concentrations until now with pharmaceutically acceptable concentrations of the active substance in the target cells of a treated mammal, especially a human being. Due to the compound according to the invention, for the first time, a medication is provided with which the supportive effects of isoflavones and isoflavone-glycosides which were only supposed until now can be reproducibly achieved. Isoflavones, isoflavone-glycosides and their pharmaceutically acceptable salts or solvates can thus for the first time be used with an exactly known, equal and reproducible structure in a pharmaceutical composition with peptides.

It is especially preferred if the quantity of the active substance and/or the quantity of the peptide alone respectively is not sufficient for the generation of the pharmaceutical and/or therapeutic effect.

Particularly preferred is a compound in accordance with the invention of one of the previously described ways for the reduction of the availability of hydrogen peroxide in a mammal, and especially a human being. Hydrogen peroxide is an initiating or at least supportive factor for the development of numerous diseases of mammals and especially humans. It has not been possible to reduce the availability of hydrogen peroxide and to eliminate its disease-supporting or disease-inducing effect with the common isoflavone compositions, especially not with human beings. It was especially not possible until now to reproducibly reduce the availability of hydrogen peroxide in the body cells of humans or other mammals in a physiologically effective way by administering the isoflavones and isoflavone-glycosides genistein, daidzein, genistin and/or daidzin. The pharmaceutical compound according to the invention remedies this for the first time.

Furthermore, a compound in accordance with the invention is preferred to one of the types described before which aims at the inhibition of thrombocyte aggregation, the prevention and/or treatment of hypertonia, hypercholesterolaemia, hyperhomocysteinaemia, diabetes mellitus, heart attack, apoplexy, arteriosclerotic angiopathy, malignant tumors and osteoporosis.

Preferably, the compounds according to the invention in the pharmaceutical composition according to the invention are given in a quantity which is sufficient in order to cause an inhibition of thrombocyte aggregation when it is administered to a mammal and especially a human being. Such an effect could not reproducibly be reached with the administration of daidzein, daidzin, genistein and/or genistin, if necessary with conventional pharmaceutical auxiliaries and carriers with the use of physiologically acceptable concentrations of said active substance and/or said active substances. Therefore, the pharmaceutical composition in accordance with the invention is advantageously suited as a substitute for conventional pharmaceutical compositions containing acetylsalicylic acid and/or clopidogrel. The pharmaceutical compositions according to the invention make it possible to reach a desired therapeutic effect without the known side-effects of conventional pharmaceutical compositions with active substances based on acetylsalicylic acid and/or clopidogrel, and especially to inhibit thrombocyte aggregation in the blood of a treated person or another mammal.

With the said substances, especially with genistein, it is possible to produce pharmaceutical compounds in accordance with the invention which are particularly suited for the inhibition of thrombocyte aggregation in a mammal as for example a human. With such compositions in accordance with the invention, in particular the undesired side-effects and therapy failures which occur in connection with the use of conventional thrombocyte aggregation inhibitors based on acetylsalicylic acid and/or clopidogrel can be prevented or at least reduced.

The therapeutic effectiveness can especially include or consist of an antioxidant effect and especially the reduction of the availability of hydrogen peroxide in a mammal, and mainly the inhibition of thrombocyte aggregation.

The pharmaceutical preparation in accordance with the invention is preferably made for oral or parenteral application. For this, the pharmaceutical composition in accordance with the invention can be given in the form of a tablet, dragée, juice or another solution. The pharmaceutical composition in accordance with the invention can preferably contain water and glucose as pharmaceutically acceptable carriers and auxiliaries. The expert will find further suited carriers and auxiliaries listed in the publication of Fiedler, H. P., Lexikon der Hilfsstoffe für Pharmazie, Kosmetik und angrenzende Gebiete [Dictionary of auxiliaries for pharmaceuticals and cosmetics and related fields], 4. edition, Aulendorf: ECV-Editio-Kantor-Verlag, 1996.

Preferably, the composition in accordance with the invention is formulated as a solid or liquid medicine, especially powder, fine powder, granulate, tablets, especially film-coated tablets, pastilles, sachets, cachets, dragées, capsules, ointments, creams, hydrogels, pastes, patches, solutions, emulsions, especially of the type oil in water, suspensions such as for example lotions, injection and infusion preparations.

Depending on the way of preparation, the pharmaceutical composition in accordance with the invention contains the compound in especially chosen quantities. Usually, a preparation in accordance with the invention will also contain iron in the quantities which are known until now and which might also depend on the way of preparation. If the pharmaceutical composition in accordance with the invention contains the compound according to the invention as an active substance, the quantity thereof will be at least 1 mg as per administered unit (e.g. as per tablet) and preferably up to 500 mg as per administered unit. Particularly preferred compositions in accordance with the invention contain a total of 10 mg up to 500 mg of the compounds as per administered unit, especially as per tablet, whereupon for the administration in the form of tablets, quantities of 100 mg to 500 mg are preferred. For the administration in a solution to be administered parenterally, the concentration of the compound according to the invention amounts to at least 0.1 mg/ml and preferably to up to 100 mg/ml, and particularly preferred to 10 to 50 mg/ml.

The new derivate glu-glu-genistein (I, X=genistein, Pep=glu-glu) can for example be transported to the cell in a considerably more effective way with the two glutamic acid residuals via a cellular peptide transport system. This guarantees that physiological effects such as an induction of the apoptosis (physiological cell death) can also be initiated by lower concentrations of glu-glu-genistein.

The invention will be further explained with the following examples and figures, whereupon the object of the invention is not restricted to those examples and figures.

### EXAMPLE 1)

### Anti-Tumor Efficiency of Glu-Glu-Genistein

The new derivate glu-glu-genistein can be transported to the cell in a considerably more effective way with the two glutamic acid residues via a cellular peptide transport system. Because of this, it can be expected that physiological effects such as an induction of the apoptosis (physiological cell death) can also be initiated by lower concentrations of glu-glu-genistein.

### Goals

The improved anti-tumor activity of the new genistein derivate glu-glu-genistein is demonstrated in comparison to genistein in 8 selected cell lines of human tumors.
In the preliminary experiments, the effects are first of all examined on the levels of apoptosis induction, cytoxicity as well as proliferation inhibition.

### Procedure

Cell lines:
   Breast carcinoma: MCF-7, MDA 435
   Colon carcinoma: SW-620, SW-480
   Malignant melanoma: A-375, SK-Mel-13
   Cutaneous squamous cell carcinoma: SCC-12, SCC-13
The cells are pre-cultivated for 2-3 weeks until an equal growth (logarithmic phase) is achieved.
For the experiments which have to be carried out, the cells are seeded in 6 well plates (200,000 cells as per well respectively). The treatment takes place after 24 hours.
In preliminary experiments, first of all, 5 different concentrations of glu-glu-genistein and of genistein are used. The effect on proliferation and cell death is assessed microscopically. Observation periods: 4 h, 8 h, 24 h, 48 h, 72 h, 6 days. The confluence as well as the share of detached cells are recorded in growth protocols. On the basis of these protocols, two suitable concentrations are selected.
If necessary, a second pilot experiment with modified conditions has to be realised.
Apoptosis assay: 24 h prior to the seeding, the cells receive a fresh growth medium. The cells are seeded in 6 well plates under the determined conditions (usually: 200,000 cells as per well). The treatment takes place after 24 h. The concentrations used and the treatment periods depend on the results of the preliminary experiments. The growth as well as the effects are controlled microscopically and logged at least 1× a day. After the expiration of the incubation period, the 6 well plates are centrifugated and the cell pellet is lysed. With the use of anti-histone as well as anti-DNA-antibodies, a sandwich ELISA is carried out which allows for a statement regarding the extent of the DNA fragmentation (Cell Death Detection ELISA, company Roche). The apoptosis is shown in relative values in relation to untreated controls.
Cytoxicity assay: 24 h prior to the seeding, the cells receive a fresh growth medium. The cells are seeded in 6 well plates under the determined conditions (usually: 200,000 cells as per well). The treatment takes place after 24 h. The concentrations used and the treatment periods depend on the results of the preliminary experiments. The growth as well as the effects are controlled microscopically and logged at least 1× a day. After the expiration of the incubation period, the cell culture supernatant is reaped. In this supernatant, the activity of the lactate dehydrogenase is determined with an encymatic reference method (LDH-release-assay, company Roche). The cytoxicity is shown in relative values in relation to untreated controls.
Proliferation assay: 24 h prior to the seeding, the cells receive a fresh growth medium. The cells are seeded in 6 well plates with a low density (usually: 50,000 cells as per well). The treatment takes place after 24 h. The assays for cell proliferation are carried out 0, 1, 2, 3, 5, 7 days after the treatment. For the determination of the number of cells, the adherent cells are fixed and coloured with crystal violet. The intensity of the colouring is determined photometrically. The cell proliferation is shown in growth curves.
For all the three experiment series (apoptosis, cytoxicity and cell proliferation), the following approaches are taken: a) untreated controls, b) genistein, concentration 1, c) genistein, concentration 2, d) glu-glu, concentration 2, e) glu-glu-genistein, concentration 1, f) glu-glu-genistein, concentration 2. All the experiments will respectively be carried out with three-fold values. The whole series of experiments is repeated 1×.
The results are evaluated with the mathematics of fuzzy systems (Leibniz system) and a final report will be prepared.

The use of representative carcinoma cell lines and the inspection of the cellular functional cascades which are essential for an anti-tumor therapy give stringent information regarding the function of glu-glu-genistein in the area of tumor therapy.

### EXAMPLE 2)

With a compound according to the invention, an inhibition of the thrombocyte aggregation which is superior to genistein can be reached because of an improved intracellular availability. This is achieved with a coupling to the dipeptide glu-glu so that gen-glu-glu in the sense of a peptidomimetic can be increasingly transported into the cells with specific oligopeptide transporters of the thrombocyte membrane.

### Experiments

Platelet-rich plasma (PRP) is mixed with genistein which is solved in DMSO in different concentrations: 0, 25, 50, 100, 200 μM. Afterwards, the collagen-induced aggregation has to be measured by aggregometry with the addition of collagen 1 ug/ml. Evaluation and identification of a genistein concentration (about 50 μM) which starts to be active
Testing of genistein vs. gen-glu-glu as described above in the area of the concentration which has previously been found to be starting to be active, for example 0, 25, 50, 75 μM. A superiority of gen-glu-glu related to the inhibition of collagen-induced aggregation is shown.

Repetition of the second experiment but with the previous addition of the indifferent dipeptide ala-ala in 100-fold concentration, i.e. 0, 2.5, 5, 7.5 mM. Now, an antagonization of the specific gen-glu-glu effect is shown since the peptide transporters are blocked by the excess of ala-ala.

### EXAMPLE 3)

### Production of Compounds According to the Invention

### C-terminal Modification:

### a) Isomer 1:

4. production of BocGlu(OtBu)Glu(OtBu)OH via solid phase synthesis on the TCP resin and elimination from the resin.
5. coupling of genistein to the protected dipeptide with HATU.
6. tBu/Boc-deprotection.
7. cleaning

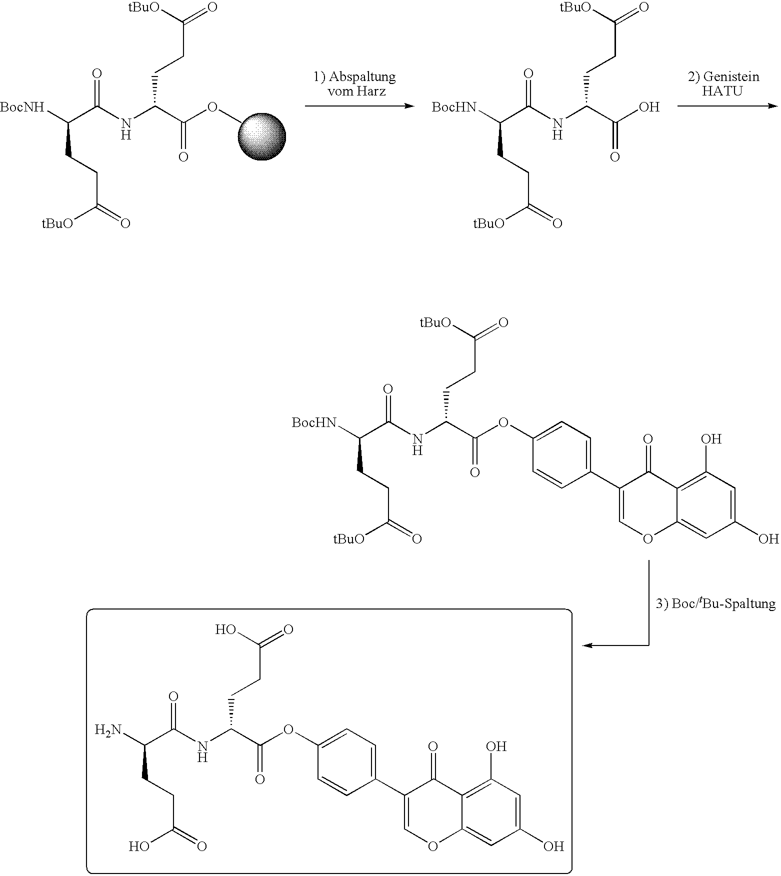

### a) Isomer 2:

4. production of BocGlu(OtBu)Glu(OtBu)OH via a solid phase synthesis on the TCP resin and elimination from the resin.
5. coupling of BocGenistein to the protected dipeptide with HATU.
6. tBu/Boc-deprotection.
7. cleaning.

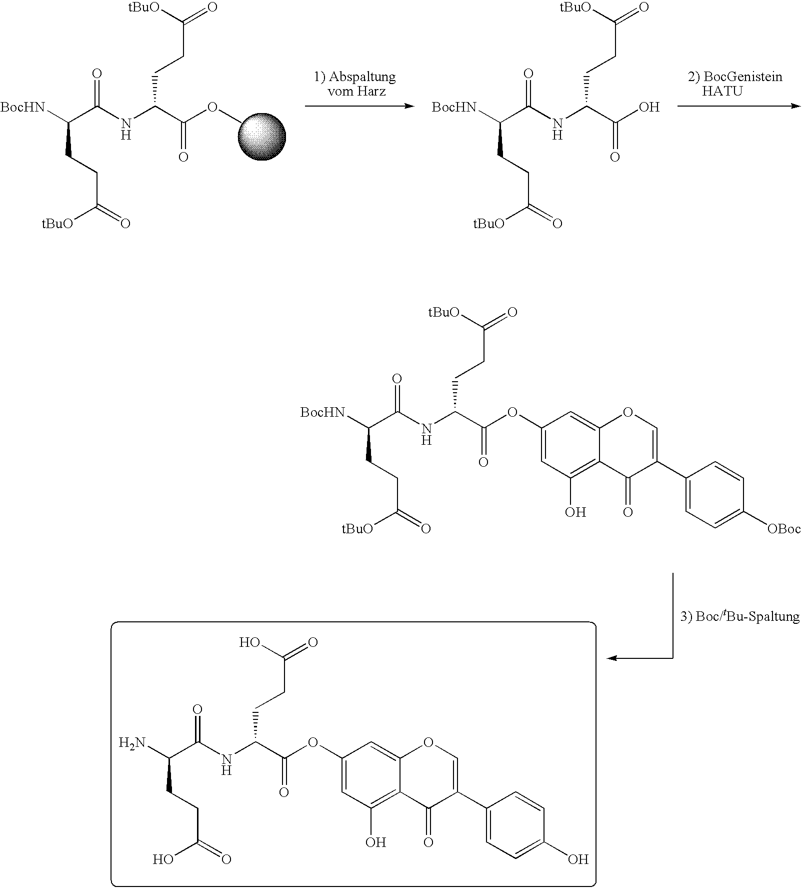

### c) Isomer 3

production of a peptide of a suitable length and sequence with at least one (OtBu)-protected side group and Fmoc-protected N-terminus via solid phase synthesis at the resin and subsequent Fmoc fission.
production of a ether-linked ethanolamine-X-conjugate.
coupling of the two fragments.
deprotection.
cleaning.

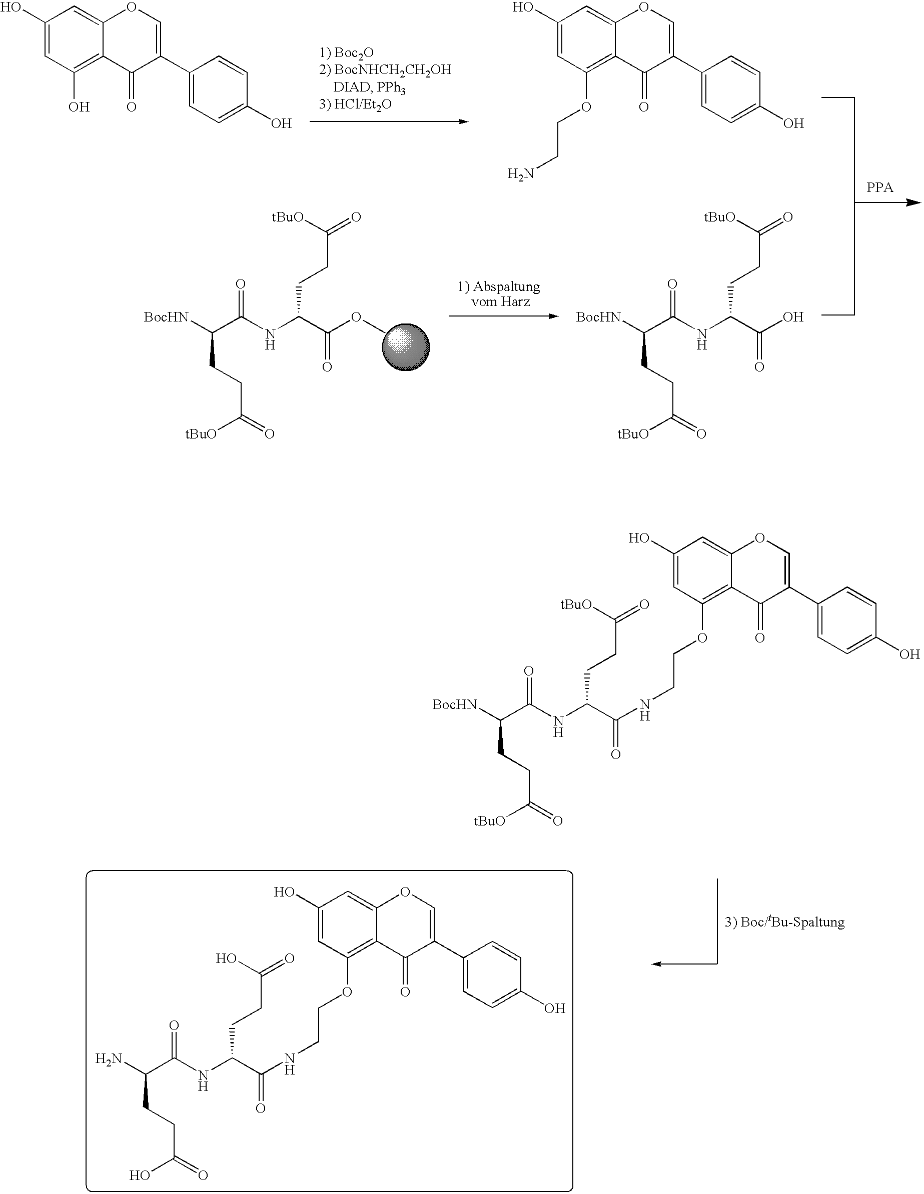

N-terminal modification:
6. production of HGlu(OtBu)Glu(OtBu)TCP via solid phase synthesis and subsequent Fmoc fission.
7. realization with BocGenistein and triphosgene.
8. elimination of resin.
9. tBu/Boc-deprotection.
10. cleaning.

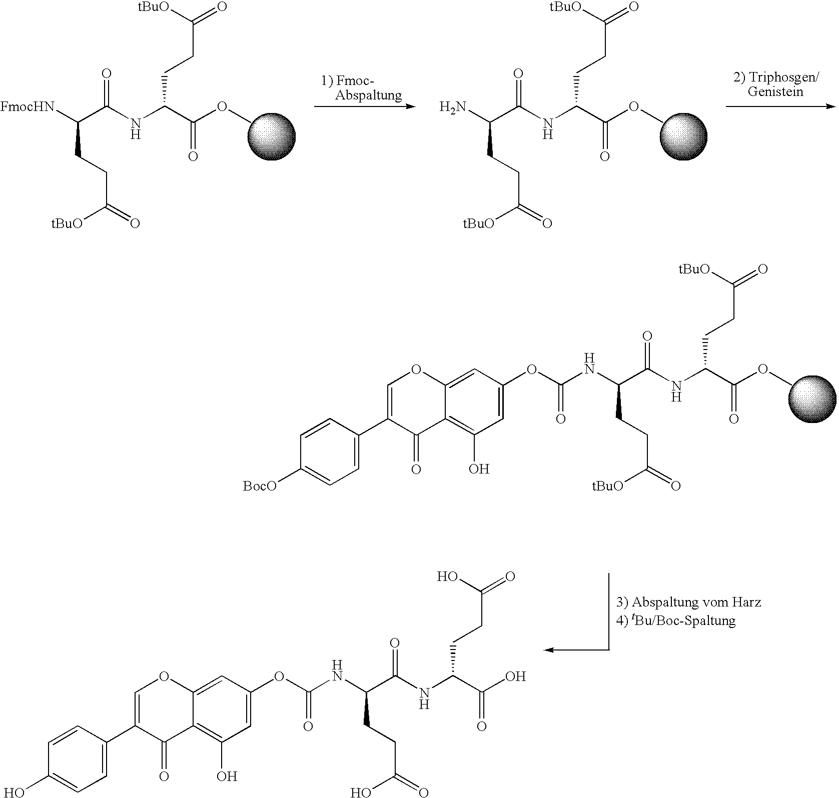

Side-chain modification:
6. coupling of FmocGlu(OAll)OH to the TCP resin.
7. A) fission of the allyl ester at the resin. B) coupling of the released acid group with BocGenistein upon utilization of HATU.
8. A) Fmoc fission. B) coupling of BocGlu(OtBu)OH.
9. elimination of resin.
10. tBu/Boc-deprotection.
11. cleaning.

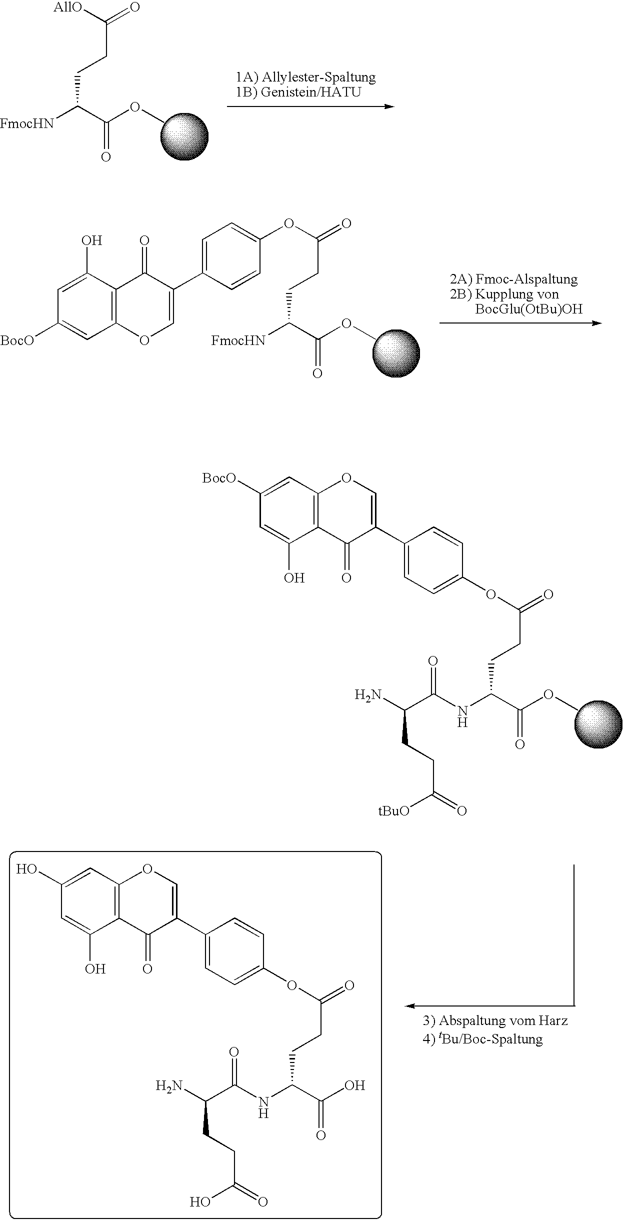

### ABBREVIATIONS

TCP resin Merrifield resin with 2-chlorotrityl linker
HATU O-(7-azabenzotriazol-1-yl)-N,N,N′,N′-tetramethyluronium-hexafluorophosphate,
   a coupling reagent
Glu Glutamic acid
Boc tert-butyloxycarbonyl, a protecting group
Fmoc 9-fluorenylmethoxycarbonyl, a protecting group
tBu tert-butyl, a protecting group

### EXAMPLE 4

### Production of 7,4′-Di-(tert-butyloxycarbonyloxy)-hydroxy-3-phenyl-4H-chromen-4-one

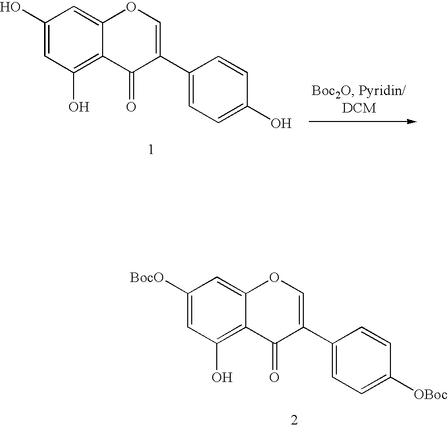

To a solution of 8.1 g (29.7 mmol) genistein 1 in DCM, 4.9 mL (2 eq.) pyridine and 13.05 g (60.7 mmol, 2.1 eq.) di-tert-butyl-dicarbonate are added, and the mixture is stirred at ambient temperature for 30 min. The solvent is extracted and the oily residue is co-evaporated twice with toluol. The accruing solid will then be dried at the high vacuum. Yield: 13 g 2 in the form of a white amorphous solid which usually consists of the three-fold transformed genistein by about 30%.

### EXAMPLE 5

### Production of 5-(2-aminoethoxy)-7-hydroxy-3-(4-hydroxyphenyl)-4H-chromen-4-one

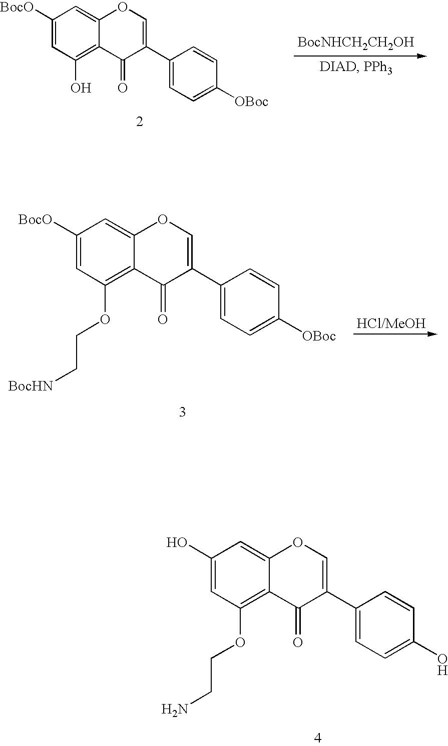

A solution of 2 (8 g, 17.01 mmol), N-Boc-ethanolamine (4.11 g, 25.5 mmol, 1.5 eq.) and triphenylphosphine (6.69 g, 25.5 mmol, 1.5 eq.) in DCM is cooled down to −15° C. and then a solution of diisopropyl azodicarboxylate (5.159 g, 25.5 mmol, 1.5 eq.) in DCM is added in drops over 5 min. The mixture is brought to ambient temperature and stirred over night. The reaction mixture is diluted with DCM, washed with water twice and then dried over sodium sulphate. The solvent is extracted and the oily residue is co-evaporated with toluol three times. The accrued residue will be solubilised with a quantity of DCM which is as low as possible and precipitated with the addition of diethyl ether.

Yield: 10.9 g of a white solid consisting of 3 and Tris-BocGenistein.

The intermediary product 3 (5.4 g) is solubilised in 10 mL methanol and treated in 30 mL of saturated methanolic HCl (1.25 mol/l) at ambient temperature. After a short time, the yellow solid which will be aspirated after 12 h starts to precipitate. The drying in the vacuum delivers pure 4 as HCl salt which will be transformed without any further cleaning.

Yield: 1.4 g 4 as a bright yellow fine powder.

### EXAMPLE 6

### Production of 5-(2-gluglu-2-aminoethoxy)-7-hydroxy-3-(4-hydroxyphenyl)-4H-chromen-4-one

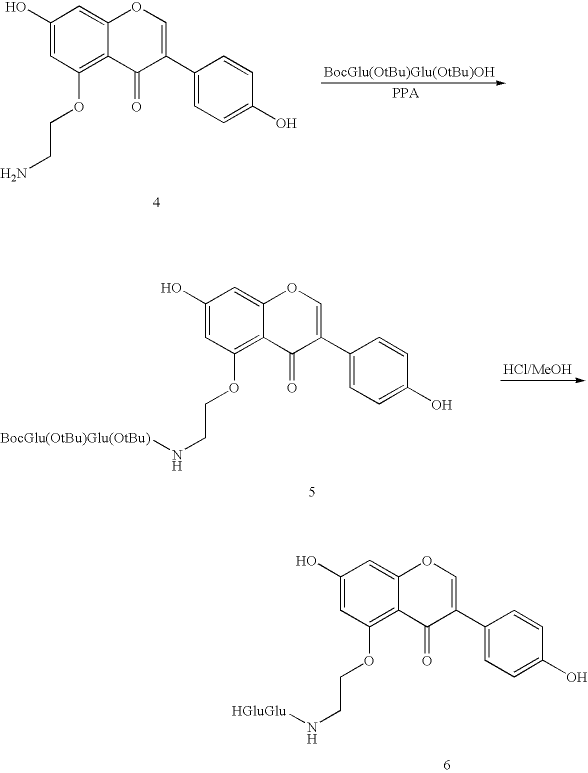

To a solution of 4 (1.22 g, 3.49 mmol) in DMF, Boc-Glu(OtBu)Glu(OtBu)OH (1.1 g, 2.59 mmol, 0.75 eq.) and PPA (50% in DMF, 2.14 mL) are added and the mixture is adjusted to pH 8-9 with the addition of N-ethyldiisopropylamine. It will be stirred for 12 h at ambient temperature.

The reaction mixture is diluted with ethyl acetate and washed with water three times, then the organic phase is dried over sodium sulphate and concentrated in the vacuum.

The residue is chromatographically cleaned (hexane/ethyl acetate 7:3→9:1→pure ethyl acetate).

Yield: 1.04 g (34%) 5 as white foam.

The intermediary product 5 (1.04 g) is suspended with 5 mL ethyl acetate and mixed with 20 mL HCl in ether (2M) so that the mixture turns yellow and clear.

After 6 h, the precipitated product is aspired, washed with ethyl acetate and diethyl ether and then finally dried in the high vacuum.

Yield: 820 mg of the chromatographically pure 6 as yellow powder.

415 mg of the raw product are solubilised in water and freeze-dried.

Yield: 291 mg 6 in the form of yellow foam.

### EXAMPLE 7

### Biological Testing

### Description of the Experiment

### Substances

The following five genistein-glu-glu conjugates were respectively tested:
Genistein-glu-glu carbamat, the coupling takes place via C-terminal over the OH group in the C⁷ position of the genistein, hereinafter referred to as C1
Ether linked with genistein-glu-glu, coupling N-terminal, methyl group as a link, bond via the OH group in the C⁵ position of the genistein, hereinafter referred to as L2
Genistein-glu-glu ether, coupling C-terminal via the OH group in the C⁵ position of the genistein, hereinafter referred to as A2
Ether linked with genistein-glu-glu, coupling N-terminal, bond via the OH group in the C⁵ position of the genistein, hereinafter referred to as L1
Genistein-glu ether, coupling C-terminal, bond via the OH group in the C⁵ position of the genistein, hereinafter referred to as A1
Native genistein
Mixture of native genistein/glu-glu

### Materials

The substances used, genistein, glu-glu, DMSO, ala-ala were provided by the company Sigma-Aldrich, Munich. For the measurement, the Platelet Aggregation Profiler Model PAP-8E of the company Bio/Data Corporation, an 8-channel aggregometer with a computerised digital curve creation, has been used.

### Pre-Analytics

After the taking of a blood sample from a large calibre vein in unbuffered citrate 10%, the whole blood which is achieved by this is centrifugated with about 1,500 RPM for 10 min. The platelet-rich plasma (PRP) which is achieved by this then has to rest for 45 min. at ambient temperature. At the same time, platelet-rich plasma for the creation of the respective blank values is gained from the same sample with common centrifugation. For the measurement, cuvettes with a volume of 0.25 ml are used.

Stock solutions of the substances to be tested, including genistein as well as the peptides ala-ala and glu-glu which are used, are prepared in a ratio of 1 mg on 1 ml, genistein as well as the tested coupling products in DMSO, glu-glu and ala-ala in H₂O.

### Preliminary Experiment

In several preliminary experiments, among others, a normal aggregation behaviour of the native sample after the addition of 1 μg/ml collagen (87% platelets aggregation, see below) as well as an indifferent behaviour of DMSO in the area of the used concentrations are shown. In the citrate blood sample, 230,000 μl of thrombocytes were measured.

### Measurements

First of all, the aggregation inhibiting effect of native genistein has been determined: With the reference (no additive), the aggregation amounted to 90% which also corresponds to the preliminary experiment. After the addition of genistein 50 μM, there was an aggregation of 82% of the platelets, in case of an addition of genistein 100 μM, the aggregation amounted to 50% and with a concentration of 200 μM, the thrombocyte aggregation was almost completely inhibited.

Principally, the results correspond to own previous studies as well as to the effects described in literature. In this, the issue of comparability has already been commented on.

According to our own specification, we now consider a concentration of 50 μM as an active concentration which starts to be effective as we expected it.

Accordingly, we examined the five genistein derivates with regard to the collagen-induced aggregation (again 1 μg/ml) with a concentration of 50 μM. The following results were achieved:

| Substance | Maximum aggregation (MA) | End point (FA) |
|---|---|---|
| C1 | 32% | 12% |
| L1 | 62% | 44% |
| L2 | 9% | 4% |
| A1 | 64% | 51% |
| A2 | 68% | 54% |

Besides a considerably more distinctive aggregation inhibiting effect of all the substances tested in comparison to the native genistein (FA 82%), a clear desegregation in the course of time is remarkable, which can be seen from the regressiveness of the curves and/or the difference between MA and FA. Moreover, the velocity of the aggregation, the so-called “slope” is considerably reduced, in particular with L2 but also with C1 in comparison to the native genistein.

For the purpose of comparison, an aggregation measurement after the addition of genistein 50 μM and glu-glu 150 μM in the sense of a mixture was carried out. The measurement was realised directly, i.e. without any relevant pre-incubation, if this is not yet immanent in the measurement method. Here also, the effect proved to be superior to genistein with MA 56% and/or FA 44% (genistein MA 85%, FA 82%).

| Substance | Maximum aggregation (MA) | End point (FA) |
|---|---|---|
| Reference | 92% | 91% |
| Genistein | 85% | 82% |
| Genistein/Glu-Glu | 56% | 44% |
| L2/Ala-Ala | 37% | 20% |

In order to support the hypothesis that the phenomenons observed relate to an improved membrane transport via specific peptide transporters on the thrombocyte membrane, another measurement of the aggregation under the influence of L2 after the previous addition of the indifferent dipeptide ala-ala which works as a substrate of the peptide transporters in the 10-fold molar excess, i.e. L2 50 μM/Ala-Ala 500 μM was realised:

This served for the partial antagonisation of the almost complete aggregation inhibition which had been observed before.

### Other Experiments

Furthermore, we examined the substance L2 for its aggregation inhibiting characteristics. First of all, the aggregation is examined after the addition of L2 in a concentration of 50 μM after the addition of collagen in increasing concentrations (observed effect reversible?):

| Substance | Maximum aggregation | End point |
|---|---|---|
| L2 50 μM/1 μg/ml collagen | 0% | 0% |
| L2 50 μM/2 μg/ml collagen | 12% | 9% |
| L2 50 μM/5 μg/ml collagen | 40% | 35% |
| L2 50 μM/10 μg/ml collagen | 50% | 50% |
| L2 50 μM/20 μg/ml collagen | 50% | 50% |

Collagen-induced aggregation after the addition of different concentrations of L2, collagen in a concentration of 1 μg/ml (dosis-effect relation?):

| Substance | Maximum aggregation | End point |
|---|---|---|
| L2 29 μM | 35% | 30% |
| L2 36 μM | 35% | 30% |
| L2 50 μM | 13% | 9% |
| L2 62 μM | 0% | 0% |

L2 50 μM after the addition of ADP 2 μM, collagen 1 μM, ristocetin 0.5, 1.0, 1.5 mg/ml, adrenaline 8 μM, arachidonic acid 0.5 μM

| L2 after the addition of | Maximum aggregation | End point |
|---|---|---|
| ADP 2 μM | | 0% |
| Collagen 1 μM | | 0% |
| Ristocetin 0.5 mg/ml | | 0% |
| Ristocetin 1.0 mg/ml | | 32% |
| Ristocetin 1.5 mg/ml | | 50% |
| Adrenalin 8 μM | | 0% |
| Arachidonic acid 0.5 μM | | 35% |

### LITERATURE

(1) Gottstein et al, British Journal of Nutrition, (2003), 89, 607-615
(2) Jacobson et al, Adv Exp Med Biol, (2002), 505, 163-71
(3) Mruk et al, Circulation (2002), 101, 324-8
(4) Guerrero et al, Journal of Thrombosis and Haemostasis, (2005), 3, 369-76
(5) Nakashima et al, Molecular Pharmacology, (1991), 39, 475-80
(6) Beretz et al, Agents Actions (1982), 12, 382-87
(7) Pignatelli et al, Blood, (1998), 91, 484-90
(8) Iuliano et al, European Journal of Biochemistry, (1994), 221, 695-704

1. Compound according to formula (I),
X-Pep (I)
whereupon
X is an isoflavone or isoflavone-glycoside, and
Pep is an amino acid or a peptide or peptide derivate with 2 to 5 amino acids,
and there is a direct covalent bond between X and Pep or the bond is created with a suitable linker system (such as ethylene glycol, ethanolamine, higher homologs thereof or the respective polyethylene glycol derivates),
as well as pharmaceutically acceptable salts or solvates of these compounds.

2. Compound according to claim 1, whereupon the isoflavone or isoflavone-glycoside contains 2-4 OH groups which are partially substituted by monosaccharides, including monosaccharides which are partially acylated with acetic acid, malonic acid, cinnamic acid, coumaric acid, caffeic acid or ferulic acid, or by disaccharides, including disaccharides which are partially acylated with acetic acid, malonic acid, cinnamic acid, coumaric acid, caffeic acid or ferulic acid, or by methyl or sulphate.

3. Compound according to claim 1 and 2, whereupon X consists of an isoflavone or isoflavone-glycoside, a substance of the general formula (II) or a pharmaceutically acceptable salt or solvate of such a substance: 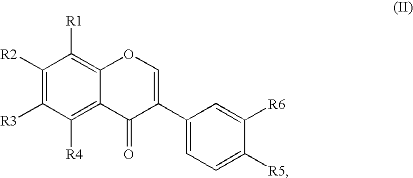
whereupon the residuals R1, R2, R3, R4, R5 and R6 can autonomously have the following meanings: hydrogen, hydroxy, methoxy or glycoside (Glc), whereupon one of the residuals 1-6 in compound I is replaced by the residual Pep (meaning explained above), preferably at the places for links R2 and R5.

4. Compound according to claim 3, whereupon the glycoside Glc shows the general formula III 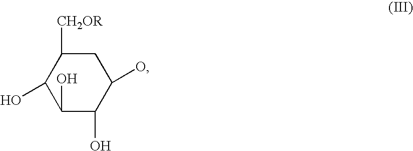
whereupon R is selected from the group consisting of hydrogen, acetyl and malonyl independent of R1, R2, R3, R4, R5 and R6.

5. Compounds according to claim 1 to 4, whereupon the isoflavone or isoflavone-glycoside is selected from the group consisting of isoflavone, daidzein, genistein, prunetin, biochanin A, orobol, santal, glycitein, pratensein, formononetin, genistin, 6″-O-malonylgenistin, 6″-O-acetylgenistin, daidzin, 6″-O-malonyldaidzin, 6″-O-acetyldaidzin, glycitin, ononin or sissotrin.

6. Compound according to claim 1, whereupon the isoflavone is genistein.

7. Compound according to claim 1, whereupon X stands for one of the following formula IV-VII

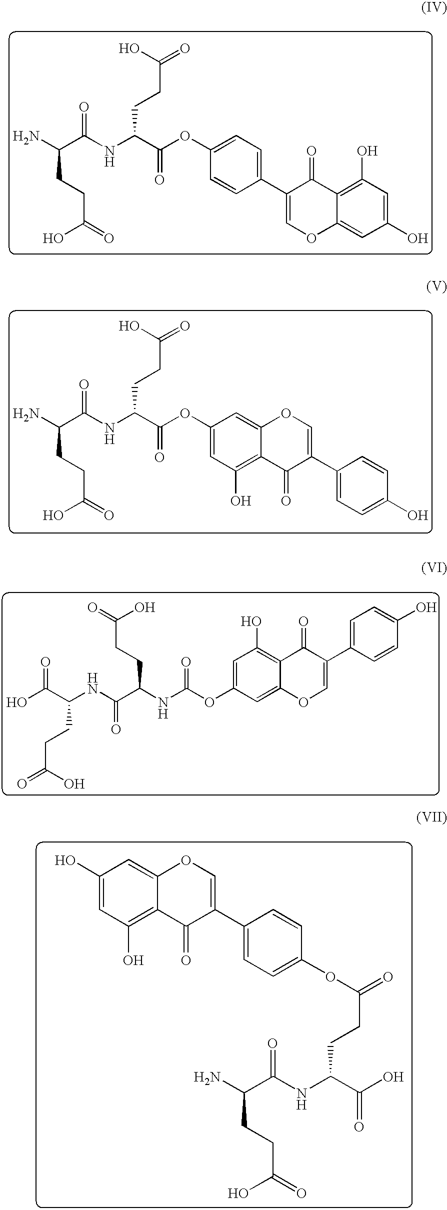

8. Compounds according to claim 1 to 7, whereupon the peptide or peptide derivate includes 2 to 5 amino acids and whereupon at least 2 amino acids comprise side-chains which are negatively loaded with pH=7.

9. Compounds according to claim 1 to 7, whereupon the peptide or peptide derivate is a dipeptide which is selected from the group of peptides with the sequences DD, EE, DE, ED; whereupon D=aspartic acid and E=glutamic acid.

10. Compounds according to claim 1 to 7, whereupon the peptide or peptide derivate is selected from the group of peptides with the sequences
DDDDD, DDDDE, DDDED, DDDEE, DDEDD, DDEDE, DDEED, DDEEE, DEDDD, DEDDE, DEDED, DEDEE, DEEDD, DEEDE, DEEED, DEEEE, EDDDD, EDDDE, EDDED, EDDEE, EDEDD, EDEDE, EDEED, EDEEE, EEDDD, EEDDE, EEDED, EEDEE, EEEDD, EEEDE, EEEED, EEEEE,
DDDD, DDDE, DDED, DDEE, DEDD, DEDE, DEED, DEEE, EDDD, EDDE, EDED, EDEE, EEDD, EEDE, EEED, EEEE,
DDD, DDE, DED, DEE, EDD, EDE, EED, EEE;
whereupon D=aspartic acid and E=glutamic acid.

11. Compounds according to claim 1, whereupon the isoflavone is genistein and the peptide or peptide derivate has the sequence EE; whereupon E=glutamic acid.

12. Compound according to claim 1 which is characterised by the fact that the covalent bond consists of a carbamat, ether or ester bond.

13. Pharmaceutical compositions including compounds according to claim 1-12.

14. Use of a compound according to claim 1-13 for the production of a pharmaceutical composition for the treatment of and/or protection against a medical indications which is selected from the following group:
cardiovascular diseases, diseases connected with an increased thrombocyte aggregation, metabolic disorders, bone diseases or cancer diseases.

15. Use in accordance with claim 14, whereupon the medical indication is selected from the following group:
leukaemia, lymphoma, melanoma, breast carcinoma, lung carcinoma, prostate carcinoma, head and neck squamous carcinoma, colon carcinoma.

16. Use in accordance with claim 14, whereupon the medical indication is selected from the following group:
hypertonia, hypercholesterolaemia, heart attack, arteriosclerotic angiopathy, apoplexy, diseases caused by an increased thrombocyte aggregation, diabetes mellitus, hyperhomocysteinaemia, malignant tumors, osteoporosis.

17. Use of a compound according to claim 1 to 12 as nutritional additive.

18. Use of a compound according to claim 1 to 12 as an additive for the protection of cells in fermenters or bioreactors.

19. Use of a compound according to claim 1 to 12 as nourishment for animals.

20. Use of a compound according to claim 1 to 12 as a pesticide.

21. Production of compounds of formula I,
including the following steps:
a) provision of an active substance selected from the group which consists of isoflavone, daidzein, genistein, prunetin, biochanin A, orobol, santal, glycitein, pratensein, formononetin, genistin, 6″-O-malonylgenistin, 6″-O-acetylgenistin, daidzin, 6″-O-malonyldaidzin, 6″-O-acetyldaidzin, glycitin, ononin and sissotrin and/or a pharmaceutically acceptable solvate or salt of the active substance, whereupon the active substance shows a protected side group, if necessary,
b) provision of an amino acid or a peptide or peptide derivate with a length of 2 to 5 amino acids or provision of an amino acid and/or a solvate or salt of the peptide or of the amino acid, whereupon at least one of the amino acids disposes of a side group which is protected and the N-terminus of the peptide or the amino acid is protected and/or the C-terminus of the peptide or of the amino acid is connected to a solid phase,
c) mixing of the active substance of a) with the active substance of b) and an activation reagent, preferably for the activation of carboxyl or hydroxyl groups.
